(19) United States
(12) Patent Application Publication
(10) Pub. No.: US 2022/0398532 A1
(43) Pub. Date: Dec. 15, 2022

(54) TWO TIER DISTRIBUTION OPTIMIZATION USING A TIME SPACE MODEL

(71) Applicant: INTERNATIONAL BUSINESS MACHINES CORPORATION, Armonk, NY (US)

(72) Inventors: Hua Ni, Chantilly, VA (US); Kriteshwar Kaur Kohli, White Plains, NY (US); Julie Starnes, Winston Salem, NC (US); Jerome Roger Luc Boyer, Santa Clara, CA (US); Trinette Ann Brownhill, Montgomery, TX (US)

(21) Appl. No.: 17/343,081

(22) Filed: Jun. 9, 2021

(51) Int. Cl. G06Q 10/08; G06Q 10/06; G06Q 10/04; G16H 40/20; F25D 11/00

(52) U.S. Cl. CPC G06Q 10/0832; G06Q 10/06313; G06Q 10/047; G06Q 10/08355; G06Q 10/087; G16H 40/20; F25D 11/003; G06Q 50/04

(57) ABSTRACT

Optimizing distribution of shipping items by receiving distribution data for a first item. The distribution data including a starting location and a destination. The distribution data also including a constraint for shipment with a second item. A time space network mode is created for tracking the first item relative to the constraint for shipment with the second item. An objection function is performed using the time space network model to optimize the distribution data by minimizing the sum of an objective function including a minimized fulfillment penalty and a minimized unmet order penalty. A delivery plan is executed with the distribution data that was optimized.

### STATEMENT REGARDING PRIOR DISCLOSURES BY THE INVENTOR OR A JOINT INVENTOR

The following disclosure(s) are submitted under 35 U.S.C. § 102(b)(1)(A): DISCLOSURE(S): IBM Garage: A Cloud Pak Show Case—Vaccine Delivery At Scale, https://ibm-cloud-architecture.github.io/vaccine-solution-main, Jerome Boyer, Rick Osowski, Sunil Dube, Hua Ni, Arnab De Adhikari, Sourav Mazumder, Stacey Ronaghan, Roger Miret Gine, Initial Publish on Jun. 11, 2020.

### BACKGROUND

The present invention generally relates to the planning for the shipping and delivery of goods, and more particularly to managing shipping and delivery of goods when the goods being shipped are delivered with reusable equipment that maintains characteristics of the goods during shipping.

In some instances, a product needed to be shipped from a producer (supplier) to a consumer (customer) needs to be shipped under specific requirements, such as temperature, that dictate that the product be shipped using specific equipment. For example, refrigerated pharmaceutical products, such as vaccines, are very sensitive to rises in temperature and the passage of time. Professionals who dispense these products know that their quality, effectiveness and safety depend, to a very large extent, on the temperature conditions at the location where they are stored, the length of time they are stored at the location before being used, as well as the total amount of time the products have been outside of refrigeration since they were manufactured.

In some examples, to maintain the temperature of the product being shipped, the product is shipped with a portable refrigerator. In these cases, not only does the distribution chain of the product have to be managed, but the distribution chain of the portable refrigerator has to checked.

### SUMMARY

In accordance with an embodiment of the present invention, a computer-implemented method is provided for optimizing distribution of an item with multi-tier requirements. In some embodiments, a computer implemented method for optimizing distribution of an item with shipping requirements is provided that includes receiving distribution data for a first item. The distribution data includes a starting location and a destination. The distribution data also includes a constraint for shipment with a second item. The method also includes creating a time space network model for tracking the first item relative to the constraint for shipment with the second item. In some embodiments, the method further includes performing an objective function using the time space network model to optimize the distribution data by minimizing the sum of an objective function including a minimized fulfillment penalty and a minimized unmet order penalty. The method may further include executing a delivery plan with the distribution data being optimized.

In some examples, the method may be employed to provide for delivery of vaccines. In this case the vaccine is the first item being delivered. The at least one handling constraint may be temperature controls. The second item meeting the requirements of the handling constraint may be a portable refrigerator unit.

In another embodiment, a system is provided for providing shipping plans. The system for optimizing distribution of shipping items may include a hardware processor; and memory that stores a computer program product. In some embodiments, when executed by a hardware processor, the memory causes the hardware processor to receive distribution data for a first item, in which the distribution data includes a starting location and a destination. The distribution data may also include a constraint for shipment with a second item. The system may also employ the hardware processor to create a time space network model for tracking the first item relative to the constraint for shipment with the second item. The system can also perform an objective function using the time space network model to optimize the distribution data by minimizing the sum of an objective function including a minimized fulfillment penalty and a minimized unmet order penalty. In some embodiments, the system can also execute a delivery plan with the distribution data being optimized.

In some examples, the system may be employed to provide for delivery of vaccines. In this case the vaccine is the first item being delivered. The at least one handling constraint may be temperature controls. The second item meeting the requirements of the handling constraint may be a portable refrigerator unit.

In yet another embodiment, a computer program product is disclosed that provides shipping plans for delivering items and coordinating shipping units with the items according to at least one handling constraint. The computer program product may include computer readable storage medium having computer readable program code embodied therewith, the program instructions executable by a processor to cause the processor to receive distribution data for a first item, the distribution data including a starting location and a destination, and the distribution data including a constraint for shipment with a second item. The computer program product can also create, using the processor, a time space network model for tracking the first item relative to the constraint for shipment with the second item. The computer program product can also perform, using the processor, an objective function using the time space network model to optimize the distribution data by minimizing the sum of an objective function including a minimized fulfillment penalty and a minimized unmet order penalty. The computer program product can also execute, using the processor, a delivery plan with the distribution data being optimized.

In some examples, the computer program product may be employed to provide for delivery of vaccines. In this case the vaccine is the item being delivered. The at least one handling constraint may be temperature controls. The second item meeting the requirements of the handling constraint may be a portable refrigerator unit.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is an illustration of an exemplary environment where a system for two tier distribution optimization is used for planning distribution of vaccine from a start location to a delivery point, in which refrigerators are coordinated with the different vaccine shipments to ensure the vaccines are maintained at the appropriate temperatures, in accordance with one embodiment of the present disclosure.

FIG. 2 is a flow chart/block diagram illustrating a method employing a two tier distribution optimization including a space model to provide for shipments of an item that are coordinated with containers for the item according to a constraint, such as keeping the temperature of the item within a specified range, in accordance with one embodiment of the present disclosure.

FIG. 3 is a block diagram illustrating one embodiment of a system that employs a two-tier distribution optimization including a space model to provide for shipments of an item that are coordinated with containers for the item according to a constraint, in accordance with one embodiment of the present disclosure.

FIG. 4 is an illustration of a time space network construction, in accordance with one embodiment of the present disclosure.

FIG. 5 is an illustration of a time space network flow balance showing the vaccine flow balance at the supplier node, in accordance with one embodiment of the present disclosure.

FIG. 6 is an illustration of a time space network flow balance showing the vaccine flow balance at the customer node, in accordance with one embodiment of the present disclosure.

FIG. 7 is an illustration of a time space network flow balance showing the container flow balance at the supplier node, in accordance with one embodiment of the present disclosure.

FIG. 8 is an illustration of a time space network flow balance showing the container flow balance at the customer node, in accordance with one embodiment of the present disclosure.

FIG. 9 is an illustration of the vaccine flow balance at the order node.

FIG. 10 is a block diagram illustrating a system that can incorporate the system that employs a two-tier distribution optimization including a space model to provide for shipments of an item that are coordinated with containers for the item according to a constraint that is depicted in FIG. 3, in accordance with one embodiment of the present disclosure.

FIG. 11 depicts a cloud computing environment according to an embodiment of the present disclosure.

FIG. 12 depicts abstraction model layers according to an embodiment of the present disclosure.

### DETAILED DESCRIPTION

The methods, systems and computer program products described herein are directed to a two tier distribution optimization using a time space model. By “two tier” it is meant that the distribution system is distributing two items that are to be coordinated together in their shipments from the starting point to the destination. For example, for large-scale vaccination operations for the COVID-19 pandemic, the vaccines are to be stored, as well as shipped, under specific temperature controls. For example, the temperature of the vaccine should be as low as −94 degrees Fahrenheit. To provide these temperatures special freezers are employed for transportation and storage of the vaccine. Both vaccine and freezer units can be potential bottlenecks in vaccine distribution. In this example, the vaccine is a first item and the freezer units are a second item that need to be coordinated in the two-tier distribution plan.

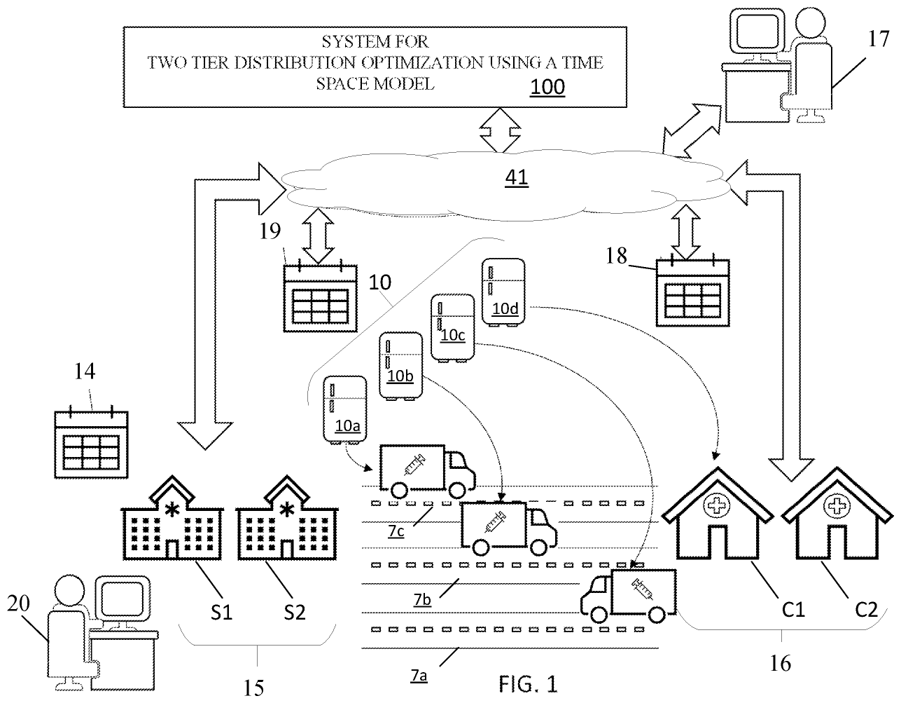

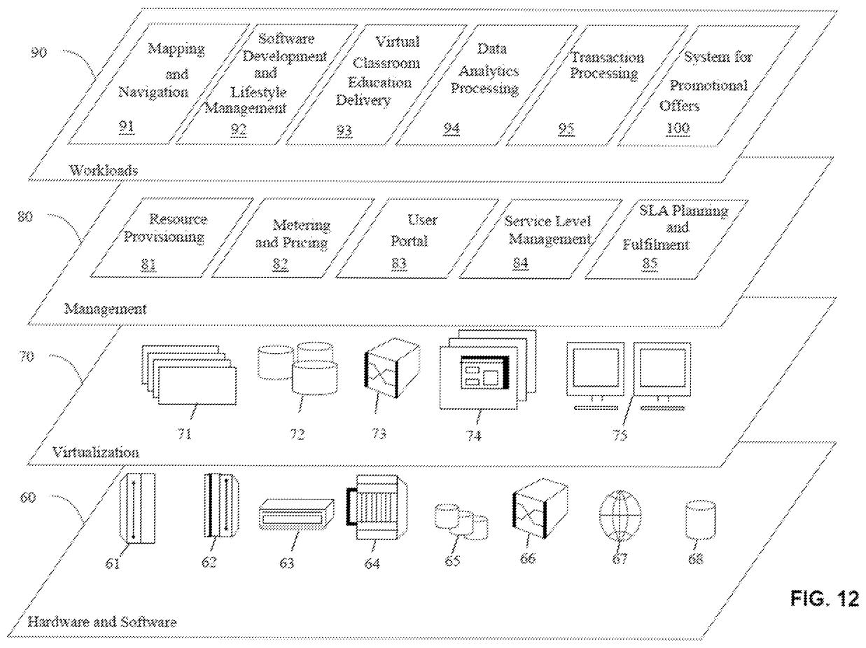

As will be described herein, an orchestration solution for decision making to protect vaccines and satisfy vaccine needs, for both the current and the future large-scale vaccination efforts is provided. The methods, systems and computer program products can provide an optimization engine and workflow to orchestrate vaccine distribution within a large-scale vaccine deployment system. An optimization model is employed using a time-space network flow construct to solve the complex fulfillment decisions for both the delivery and storage of the vaccine as well as the availability of freezers used in maintaining vaccine temperature. The optimization model can provide decisions on from which plant/warehouse to supply the vaccine, and the optimization model can also provide for dynamically repositioning of the freezers while in-motion, e.g., traveling between different locations between the start location for a delivery, the destination for a delivery and locations therebetween. The optimization model can minimize timing deviation from customer request while minimizing the logistics cost. It is noted that although the present disclosure describes examples for shipping with constraints to include freezers in the shipping plans to maintain the temperature of vaccines that the present disclosure is not limited to only this example. For example, other applications are equally applicable to the methods, systems and computer program products of the present disclosure. One application for the shipping system including the optimization model is the shipping of food items that require temperature control. Another application is to provide schedules for shipping multiple items from multiple start points in a single shipment. The methods, systems and computer program products that provide for a two tier distribution optimization using a time space model are now described with greater detail with reference to FIGS. 1-12.

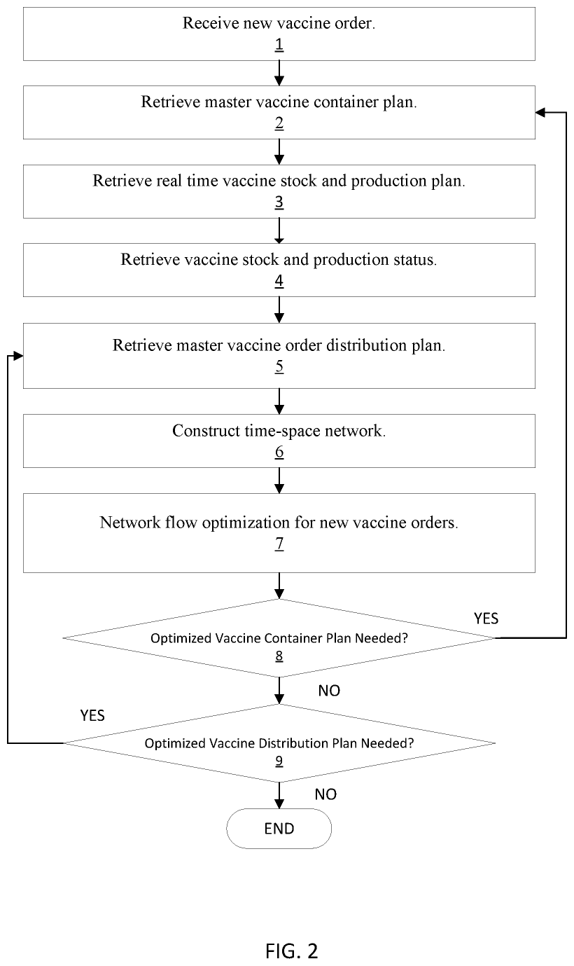

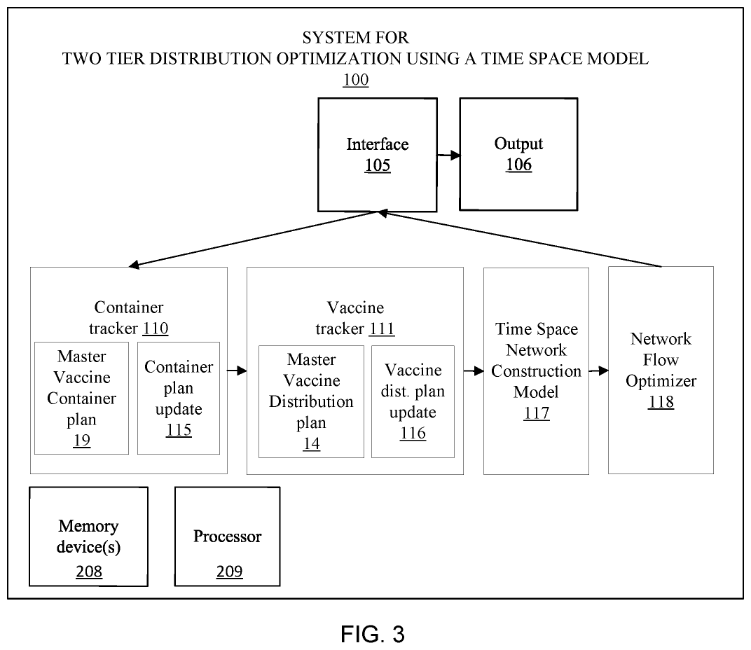

FIG. 1 is an exemplary environment, where a system 100, e.g., system 100 for two tier distribution optimization, is used for planning distribution of vaccine from a start location 5 to a delivery point 16, in which refrigerators 10 are coordinated with the different vaccine shipments 7a, 7b, 7c to ensure the vaccines are maintained at the appropriate temperatures. FIG. 2 is a flow chart/block diagram illustrating a two-tier distribution optimization method including a space model to provide for shipments of an item that are coordinated with containers for the item according to a constraint, such as keeping the temperature of the item within a specified range. FIG. 3 illustrates one embodiment of a system that employs visual artefacts including pictograms to search source code, which in some embodiments may be employed with the method described in FIG. 1. It is noted that the embodiments described with reference to FIGS. 1-3 are specific to vaccine distribution. It is noted that the present disclosure is not intended to be limited to only this example. For example, the vaccine can be substituted in the first item for food. In this example, the temperature of the food would be controlled by the containers during shipping. In another example, the constraint provides that the multiple products originating from different warehouses need to be in a single shipment to a delivery point.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

FIG. 1 is an exemplary environment, where a system 100, e.g., system 100 for two tier distribution optimization, is used for planning distribution of a first item, e.g., vaccine, from a start location 5 to a delivery point 16, in which a second shipping item, e.g., refrigerators 10, is coordinated with the different shipments 7a, 7b, 7c to ensure the vaccines are maintained at the appropriate temperatures. For example, some vaccines, such as vaccines suitable for COVID-19, have care instructions for ultra-low temperatures throughout the vaccine distribution process, e.g., such as low as −94° F. For large scale vaccine distribution, the availability of special vaccine containers can pose a critical limitation on the vaccine supply chain on top of anticipated production limitations. In some embodiments, a streamlined vaccine distribution optimization process, and an innovative mathematical optimization model that optimizes both the vaccine distribution plan 18 and a vaccine container reposition plan 19 is provided. This will expand the capacity of vaccine distribution 18 and the efficiency of the operations. The methods, systems and computer program products described herein can reduce the delay and errors due from human schedulers and improve the on-time delivery of the vaccines.

The system 100 provides an optimization engine (network flow optimizer 118) and workflow to orchestrate vaccine distribution within a large-scale vaccine deployment system. The vaccine deployment system can include a vaccine stock and production plan 14. The vaccine stock and production plan 14 considers the output of the different vaccine manufacturers, e.g., suppliers S1, S2. The vaccine manufacturers S1, S2 can provide the start location 15 for distribution. The vaccine stock and production plan 14 also includes timing information. More specifically, the vaccine stock and production plan 14 can provide a schedule of when vaccine is ready for shipment, and how many units for the vaccine is available for shipment. Multiple vaccine manufacturers S1, S2 can be included in the production plan 14. The multiple vaccine manufacturers S1, S2 have addresses which provide the start 15 for the delivery. The vaccine stock and production plan 14 can be updated in real time.

The vaccine deployment system 100 can also include a vaccine container plan 19. The vaccine container plan 19 provides information on the location of vaccine containers. The containers may be refrigerated containers 10. The containers 10 may be tracked according to the routes 7a, 7b, 7c for delivery. For example, a first set of containers 10a may be configured to be present at the starting location for shipment 7c. In determining when the first set of containers 10a are available for the starting location 10a, the containers 10b, 10c are also considered, as well as the location of the containers 10d that may be handling the vaccine at the destination 16. The information may all be elements of the vaccine container plan 19.

The vaccine deployment system 100 can also include a master vaccine order distribution plan 8. The master vaccine order distribution plan 8 includes data for the order of the vaccines. The order information includes the number of units of vaccine being ordered, and the destination address 16.

The vaccine deployment system 100 can be in communication with the each of the master vaccine order distribution plan 8, the vaccine container plan 19, the vaccine stock and production plan 14, the manufacturers (suppliers S1, S2) at the start location 15, the customers (C1, C2) at the delivery point 16 and the refrigerators 10 may be in communication with the system 100 through a network 41 that can be provided through the internet.

In some embodiments, the methods, systems and computer program products, the vaccine orders are fulfilled, e.g., in accordance with the vaccine order distribution plan 8, over time to account for the timing of the delivery needs and the time of container repositioning movement and vaccine transport. The vaccines at the supplier sites S1, S2 are also becoming available over time following production or replenishment schedule, and vaccine transports, e.g., over the shipping routes 7a, 7b, 7c, are restricted by the refrigerator 10 and/or container availability. To more effectively model this complex distribution, the supply and customer network needs to be transformed into a time-space network that is provided by the vaccine deployment system 100. The distribution optimization problem can then be modeled as a network flow problem. The methods, systems and computer program products of the present disclosure is not just a generic supply chain optimization that considers logistical issues, supply & demand planning or inventory allocation. The vaccine deployment system considers specific vaccine characteristics as related to delivery and fulfillment. For example, the vaccine characteristics being considered by the system 100 may be temperature control, which can be handled through the usage of the refrigerators 10.

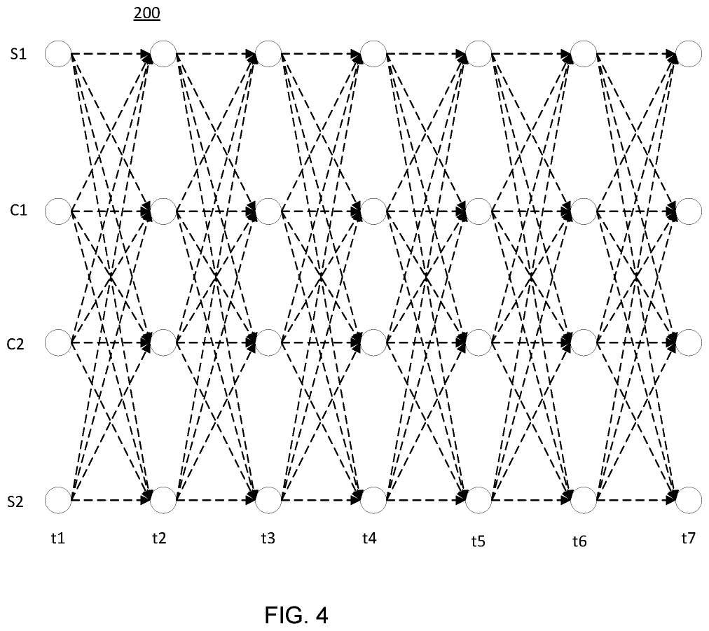

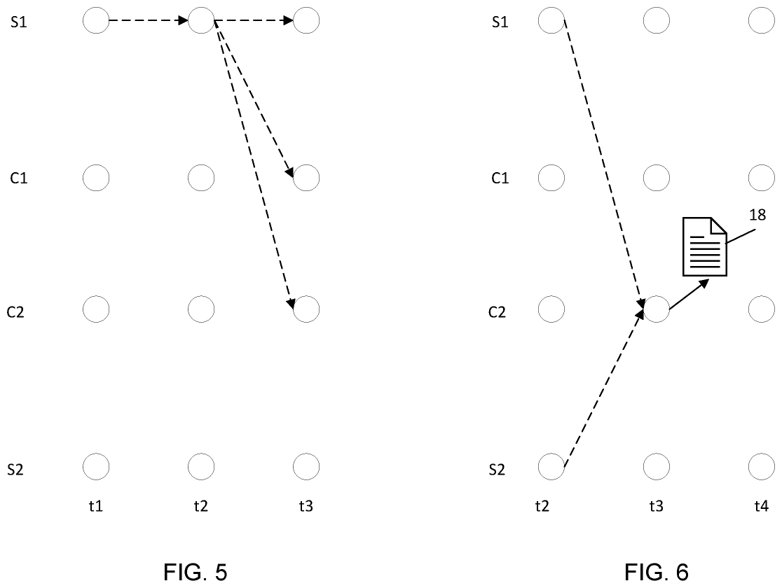

FIG. 1 is an illustration of an exemplary environment where a system 100 for two tier distribution optimization is used for planning distribution of vaccine from a start location 15 to a delivery point 16, in which refrigerators 10 are coordinated with the different vaccine shipments 7a, 7b, 7c to ensure the vaccines are maintained at the appropriate temperatures. FIG. 4 is an illustration of a time space network construction 200 that can be used for the distribution optimization provided in the method illustrated in FIG. 1. FIGS. 5-9 are illustrations of a time space network flow balance that can be used for the distribution optimization provided in the method illustrated in FIG. 2. The optimization model seeks to minimize the logistics costs of vaccines and refrigerator containers to fulfill the vaccine orders within the delivery windows. The logistics cost may include the shipping costs, both fixed costs per shipment, and variable costs per refrigerator containers, as well as the storage costs of refrigerators 10 at the supplier sites 15 and customer sites 16 The fulfillments are subjected to the availability of both vaccines and refrigerator 10 containers. The orders may have a different priority levels so that when the resources are limited, the more critical orders will be given priority to fulfill. After optimization, the system 100 can process the results to construct the optimized plans for both the vaccines and refrigerator containers. The vaccine plan 8 will specify which supplier site will provide how much quantity of vaccines for the order to be delivered at what time. The refrigerator plan 19 is supporting the vaccine plan 8 so that then vaccine moves, there will be enough refrigerators to transport.

In some embodiments, the method may begin with block 1 of the method depicted in FIG. 2, which can include receiving an order for a vaccine. Referring to FIG. 1, in some embodiments, the order for the vaccine may be entered by a user 17 that interacts with the system 100 via the user interface (UI) of a computer, e.g., desktop computer/workstation, and/or the user interface (UI) of a mobile device, to enter an order for a vaccine delivery. The user 17 enters the type of vaccine, amount of vaccine, delivery point for the vaccine, and a requested shipping date. The user 17 can also enter priority levels so that when the resources are limited, the system 100 can give more critical orders priority to fulfill. Referring to FIG. 3, the system 100 may include an interface 105 for receiving the order request. The interface 105 can also receive an input from the supplier S1, S2, e.g., from a representative 20 of the supplier S1, S2.

Referring to FIG. 2, in a following step, the master vaccine container plan 19 is received at block 2. The master vaccine container plan 19 is continually updated. The vaccine container plan 19 includes a real time record of the location of containers 10a, 10b, 10c, 10d relative to vaccine shipping. For example, the vaccine container plan 19 may track a first container 10a that is being loaded with a new shipment 7c of vaccine that is being distributed from one of the manufacturers S1, S2. The vaccine container plan 19 may track a second container 10b that is on route from the manufacturers S1, S2 to the customer C1, C2. The vaccine container plan 19 may also track a third container 10c as the shipping service is returning the container 10c back to the manufacturer after delivery. In some examples, the vaccine container plan 19 may also track a fourth container 10d that is maintaining temperature of vaccine while at the customer and being stored prior to use.

Referring to FIG. 3, the system 100 may include a container tracker 110. The container tracker 110 includes both memory for the master vaccine container plan 19, and a container plan updater 115 for tracking the location of the containers, e.g., refrigerators, and updating the master vaccine container plan 19. In some embodiments, the refrigerators 10 may be tracked using GPS signal. In other embodiments, tracking of the refrigerators 10 can be accomplished by manual recording the location of the refrigerators during the traveling between the manufacturer S1, S2 and the customer C1, C2. Upon receipt of data indicating that a refrigerator 10 has been moved, the container plan updater 115 can update the master vaccine container plan 19.

At block 3 of FIG. 2, the method can further include retrieving real time vaccine container status. In some embodiments, the step can employ the container tracker 110. Referring to FIG. 3, in some embodiments, the container tracker 110 employs GPS to determine the transportation state and/or location of the refrigerators 10. Using the container plan updater 115, the master vaccine container plan 19 can be updated.

At block 5 of FIG. 2, the method can continue with retrieving the vaccine stock and production plan 14. The vaccine stock and production plan 14 includes data illustrated the amount of vaccine that is currently available at each manufacturer S1, S2, as well as estimates when additional vaccine will be available in the future. The vaccine stock and production plan 14 may be updated in real time. Referring to FIG. 1, the vaccine stock and production plan 14 may be entered into the system 100 by personnel 20 from the manufacturer S1, S2.

Referring to FIG. 3, the system 100 may include a vaccine tracker 111. The vaccine tracker 111 includes both memory for the vaccine stock and production plan 14, and a vaccine stock and production plant updater 116 for tracking the updating vaccine availability information. In some embodiments, the updates are provided by personnel 20 from the manufacturer S1, S2. The updating process can also be automated.

Referring to FIG. 2, the master vaccine container plan that is retrieved at block 2 and the master vaccine order distribution plan 8 that is retrieved at block 3 may be updated in real time. The updates can be provided using a constraint time space network at block 6 in combination with a network flow optimization for new vaccine orders that is conducted at block 7.

FIG. 4 is an illustration of a time space network construction 200 in accordance with some embodiments of the present disclosure. In the example depicted in FIG. 4, the time space network 200 is considering two customers (C1, C2) and two suppliers (S1, S2). The customers (C1, C2) correspond to the customers C1, C2 that provide the delivery destinations 16 in the illustration depicted in FIG. 1. The suppliers (S1, S2) correspond to the manufacturers S1, S2 that are provide the start point 15 for delivery in FIG. 1. Each node in the time space network 200 represents a location in time, e.g., t1, t2, t3, t4, t5, t6, t7. Each arc in the time space network 200 represent a viable flow in material. In the example, in which the item being shipped is a vaccine, and the container that provides the special performance required for the item is a refrigerator, the viable material flows can include: 1) empty vaccine containers being repositioned, 2) vaccines being distributed within the refrigerated containers, and 3) vaccines becoming available after production. Using the time space network construction, optimization of deliveries can be provided by identifying the material flows that carriers the vaccines and vaccine containers to the order destination node (for the customer) at the desired delivery time.

FIGS. 5-9 are illustrates of time space network flow balances. The nodes are at different points of time, e.g., t1, t2, t3, t4. Arcs between the different nodes represent movement of materials, e.g., vaccine or containers, such as refrigerated containers, for moving the vaccine. FIG. 5 is an illustration of a time space network flow balance showing the vaccine flow balance at the supplier node. More specifically, FIG. 5 illustrates the network flow from the first supplier S1, and illustrates inventory being available at times nodes t2 and t3. These arcs extend from t1 to t3 and indicate inventory available. FIG. 5 also illustrates shipments to consumers C1 and C2 from the second time node t2 to the third time node t3. These arcs extend from the supplier S1 to the first and second customers C1, C2 from the second time node t2 to the third time node t3.

FIG. 6 is an illustration of a time space network flow balance showing the vaccine flow balance at the customer node. In this example, the customer node is C2. From time nodes t2 to t3 transports from suppliers S1 and S2 are made to the customer C2. This is in accordance with a delivery order 8.

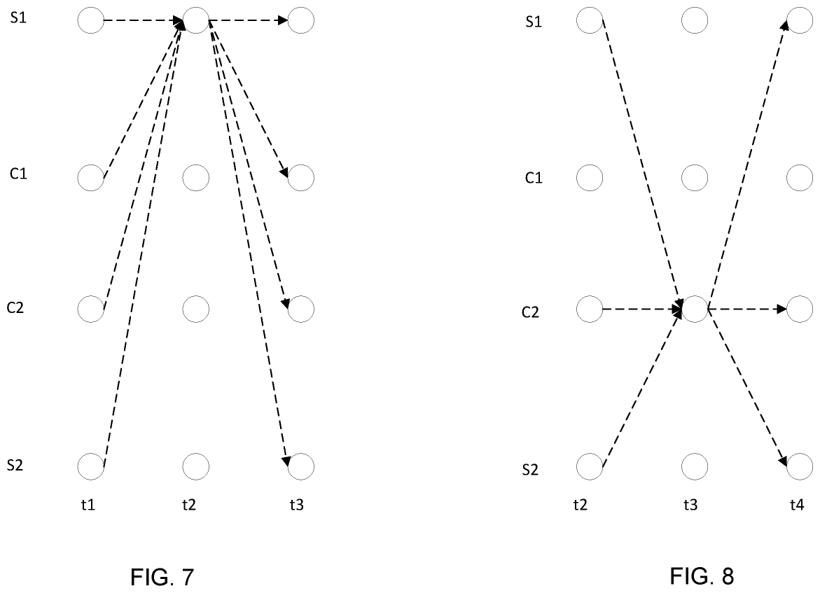

FIG. 7 is an illustration of a time space network flow balance showing the container flow balance at the supplier node. In this example, the time space network flow balance is from the perspective of a first supplier S1. From the time nodes t1 to t2, containers stay at the first supplier S1. Additionally, from time nodes t1 to t2, containers are repositioned from the customers C1, C2 and the second supplier S2 to the first supplier. From time nodes t2 to t3, the containers are again repositioned. In some instances, the repositioning of the containers 10 can be through shipments accompanying the vaccine to customers C1, C2. In some instances, containers 10 can be repositioned to other suppliers, such as the reposition illustrated by the arc from the first supplier S1 to the second supplier S2. The arcs to the supplier S1 nodes are repositioning of the containers 10. The arcs from the supplier S1 to customers C1, C2 are transports with vaccine. The arc from a first supplier S1 to a second supplier S2 is a repositioning of the containers 10. In some instances, portions of the containers 10 may be retained at the first supplier S1. This is for use in subsequent shipments.

FIG. 8 is an illustration of a time space network flow balance showing the container flow balance at the customer node, i.e., second customer node C2. In this example, from time node t2 to time node t3, containers may be transported to the customer C2 from the suppliers S1, S2. This can be an indication of delivery of vaccine in combination with containers 10. From node t3 to t4, the containers 10 may be repositioned from the customer C2 back to the suppliers S1, S2. Some containers can stay at the customer C2 from time node t2 to time node t4.

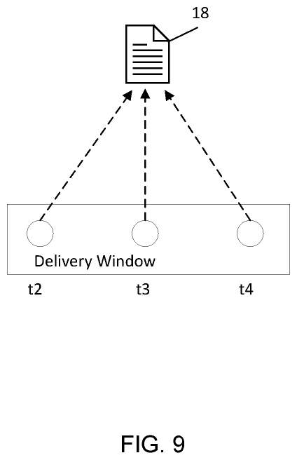

FIG. 9 is an illustration of the vaccine flow balance at the order node 8. The vaccine flow balance illustrates that there are three deliveries being made at the time nodes t2, t3, t4.

Referring to FIGS. 2 and 3, at block 6 the method constructs a time space network 200 using the data retrieved in blocks 2, 3, 4 and 5. The system 100 may include a time space network construction module 117. The time space network construction module may include at least one hardware processor 209 and memory 208 for the purposes of converting the data that is received by the system and converting that data into a time space network similar to those depicted in FIGS. 4-9.

Referring to FIG. 2, the method may continue by employing the space network construction in performing a network flow optimization for new vaccinated orders, such as the new vaccine order that is entered into the system at block 1. Referring to FIG. 3, in some embodiments, the system 100 may include an optimizer 118 (network flow optimizer) that employs the time space network. In some embodiments, the flow balances depicted in FIGS. 5-9 can be used to regulate the material flows to ensure the optimizer 118 identify visible paths with the respectable available materials.

The optimizer 118 of the system 100 includes memory for storing a set of instructions to be executed by at least one hardware processor 209 so that the optimizer can employ an objective function that can minimize the sum of 1) the logistics fixed cost for vaccine delivery, 2) the logistics variable costs for vaccine delivery, 3) fulfillment penalties in failure to deliver vaccine, and 4) unmet order penalty for a failure to deliver vaccine. One embodiment of the objective function is equation 1 (EQ1), as follows:

min Σ_{αϵA}_{^{R}}L_{α}^{FX}Z_{α}+Σ_{αϵA}_{^{R}}L_{α}^{RF}y_{α}+Σ_{oϵN}_{^{o}}Σ_{c:(c,o)ϵA}_{^{co}}P_{O}F_{(c,o)}^{v}x_{(c,o)}+Σ_{oϵN}_{^{o}}P_{O}Qwₒ EQ1.

The objective function indicates how much each variable contributes to the value to be optimized in the problem.

In the objective function for equation 1 (EQ1), the decision variables are as follows: xₐ=(linear) number of vaccine flowing through arc α, αϵA^{V}.

yₐ=(integer) number of vaccine containers (refrigerators) flowing through arc α, αϵA^{V}.
zₐ=(binary) number of vaccine containers (refrigerators) flowing through arc a, aϵA^{v}.
wₒ=(linear) unmet demand at order node o, oϵN^{o}.

The constraints to be used with the objection function in equation 1 (EQ1) include a capacity constraint (CON_CAP) on supplier (S1, S2) to consumer (C1, C2) arcs, as illustrated in the time space network flows depicted in FIGS. 4-9. The CON-CAP constraints are in equation 2 (EQ2) as follows:

x_{α}≤V_{Y}_{_{α}}αϵA^{sc} EQ2.

The constraints to be used with the objection function in equation 1 (EQ1) include a balance constraint (CON_R_BAL_SUPPLIER) at the supplier (s1, s2) for vaccine containers, e.g., refrigerators 10. The CON_R_BAL_SUPPLIER constraint is in equation 3 (EQ3) as follows:

Rₛ+Σ_{m:(m,s)ϵA}_{^{R}}yₘₛ=Σ_{n(s,n)ϵA}_{^{R}}yₛₙ EQ3.

The constraints to be used with the objection function in equation 1 (EQ1) include a balance constraint (CON_V_BAL_CUSTOMER) at the customer (c1, c2) for vaccine. The CON_V_BAL_CUSTOMER constraint is in equation 4 (EQ4) as follows:

R_{c}+Σ_{m:(m,c)ϵA}_{^{R}}y_{mc}=Σ_{n:(c,n)ϵA}_{^{R}}y_{cn} cϵÑ^{c} EQ4.

The constraints to be used with the objection function in equation 1 (EQ1) include a balance constraint (CON_V_BAL_SUPPLIER) at the supplier (s1, s2) for the vaccine. The CON_R_BAL_SUPPLIER constraint is in equation 5 (EQ5) as follows:

Vₛ+x_{s̅s}=_{n:(s,n)ϵA}_{^{v}} xₛₙ sϵÑ^{s} EQ5.

The constraints to be used with the objection function in equation 1 (EQ1) include a balance constraint (CON_V_BAL_CUSTOMER) at the supplier (s1, s2) for the vaccine. The CON_V_BAL_CUSTOMER constraint is in equation 6 (EQ6) as follows:

Σ_{m:(m,C)ϵA}_{^{v}} x_{mc}=Σ_{o:(c,o)ϵA}_{^{CO}}x_{co} EQ6.

The constraints to be used with the objection function in equation 1 (EQ1) include a balance constraint (CON_V_BAL_ORDER) at the order node for the vaccine. The CON_V_BAL_ORDER constraint is in equation 7 (EQ7) as follows:

Σ_{c:(c,o)ϵA}_{^{CO}} x_{co}=Dₒ−wₒ oϵN^{o} EQ7.

The constraints to be used with the objection function in equation 1 (EQ1) include a shipping notification (CON_SHIPPING_LANE_LB) on lane (Lower Bound). The CON_SHIPPING_LANE_LB constraint is in equation 8 (EQ8) as follows:

y_{α}≤M_{Z}_{_{α}}αϵA^{R} EQ8.

The constraints to be used with the objection function in equation 1 (EQ1) include a shipping notification (CON_SHIPPING_LANE_UB) on lane (Upper Bound). The CON_SHIPPING_LANE_UB constraint is in equation 9 (EQ9) as follows:

z_{α}≤y_{α}αϵA^{R} EQ9.

The network flow optimization model for vaccine order optimization that employs the objective function in equation 1 (EQ1) may include a set of nodes and arcs consistent with time space network flows depicted in FIGS. 4-9. The set of nodes (N) in the time space network, m, nϵN may include the following:

N^{S}=Nodes corresponding to suppliers (supplier/day), sϵN^{s}.
Ñ^{S}=Nodes corresponding to suppliers, except for the last day on horizon, sϵN^{s}.
N^{C}=Nodes corresponding to customers (supplier/day), cϵN^{c}.
Ñ^{C}=Nodes corresponding to customers, except for the last day on horizon, cϵN^{c}.
N^{O}=Nodes corresponding to orders, oϵN^{o}. [symbol]
=Predecessor node to node m by the same entity (supplier/customer).

The set of arcs (A) in the time space network, α=(m, n)ϵA may include the following:

A^{SC}=Set of arcs from suppliers to customers.
A^{SS}=Set of arcs between suppliers.
A^{CO}=Set of arcs from customers to orders.
A_{CS}=Set of arcs from customers to suppliers (for refrigerator relocation).
A^{S}=Set of arcs from supplier to itself in the next time period.
A^{C}=Set of arcs from customer to itself in the next time period.
A^{R}=Set of arcs where the refrigerators flow, A^{R}=A^{SC}U A^{SS}U A^{S} U A^{C}.
A^{V}=Set of arcs where the vaccines flow, A^{V}=A_{SC}U A^{CO} U A^{S}.

The data for the constraints and objective function for equation (EQ1) include:

Dₒ=Vaccine order demand at order node o, oϵN^{o}.
Pₒ=Priority value for order node o, oϵN^{o}.
Vₛ=Vaccines becoming available (current inventor+new production) at supplier node, s, sϵN^{s}.
Rₙ=Refrigerators available at node n, nϵ(N^{S} U N^{C}).
L_{α}^{RF}=Logistics cost per refrigerator on arc α, αϵA^{R}.
L_{α}^{FX}=Logistics cost of shipping on arc α, αϵA^{R}.
F_{α}^{V}=Order fulfillment penalty/cost on arc α, αϵA^{CO}.
U=Number of vaccines carried by refrigerator container.
Q=Penalty value for unmet demand
M=number of refrigerator container in system.

At block 7 of the method depicted in FIG. 2, a network flow optimization for new vaccine orders employs the network flow optimization model for vaccine order optimization and the objective function in equation 1 (EQ1) consistent with the objective variables, constraints, definitions for arcs and nodes and data that is described above to provide an optimized delivery schedule of vaccine coordinated with refrigerated containers. The objective function can minimize the sum of 1) the logistics fixed cost for vaccine delivery, 2) the logistics variable costs for vaccine delivery, 3) fulfillment penalties in failure to deliver vaccine, and 4) unmet order penalty for a failure to deliver vaccine.

In some embodiments, to perform the network flow optimization for new vaccine orders at block 7, the system 100 may include a network flow optimizer 118. The network flow optimizer 118 includes memory 208 for storing instructions for the objective function in equation 1 (EQ1) consistent with the objective variables, constraints, definitions for arcs and nodes and data that is described above to provide an optimized delivery schedule of vaccine coordinated with refrigerated containers. The network flow optimizer 118 also includes a hardware processor 209 for performing the calculations and analysis in providing the optimized delivery schedule. The optimized delivery schedule performs deliveries for vaccines and coordinated refrigerator containers at minimum sum of 1) the logistics fixed cost for vaccine delivery, 2) the logistics variable costs for vaccine delivery, 3) fulfillment penalties in failure to deliver vaccine, and 4) unmet order penalty for a failure to deliver vaccine with the appropriate refrigerated containers.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), FPGAs, and/or PLAs. These and other variations of a hardware processor subsystem are also contemplated in accordance with embodiments of the present invention.

At blocks 8 and 9 of FIG. 2, the method may continue with creating (update) an optimized vaccine container plan at block 8 and/or creating (update) an optimized vaccine container plan. The optimized vaccine container plan is fed back into block 2 of the method for consideration with a subsequent vaccine order. Similarly at block 9, the method may include creating (update) an optimized vaccine distribution plan. The optimized vaccine distribution plan is fed back into block 5 of the method for consideration with a subsequent vaccine order.

In some embodiments, the delivery plan with the distribution data being optimized in accordance with the method described in FIG. 2 may be executed to deliver vaccine with corresponding freezers to customers C1, C2 at the delivery address 16. The delivery plan may be an output 106 of the system depicted in FIG. 3.

As noted, although the methods, systems and computer program products have been described herein for providing deliveries of vaccines coordinated with refrigerators 10, the present disclosure is not limited to only this example. For example, in some embodiments, the methods systems and computer program products may be used in accordance with a cold chain optimized delivery & allocation plan. Cold chain food delivery employs temperature control throughout the distribution process. To employ the methods, systems and compute program products described herein, cold chain food is substituted for the vaccines in the above description.

The patent described in this disclosure provides a streamlined cold chain distribution optimization process and an innovative optimization model that optimizes both the cold chain product distribution plan and refrigerated container reposition plan.

The methods, systems and computer program products may also be used for delivering multiple products originating from different warehouses need to be in single shipment to a delivery point. Fulfillment (pulling from multiple stocking locations) can be optimized based on source locations, delivery point locations, specialized container availability and location, and logistics restrictions. Offshore platforms with a limited windows for shipment of critical supplies, a limited number of specialized containers, and multiple current location of those containers are also scenarios suitable for use with the methods, systems and computer program products that are described herein. Moving operation/delivery points can also benefit from optimization of fulfillment plans and logistic/distribution plans for the mobile delivery point (when it must move to another location). Ships (mobile delivery point) require fuel along their journey, that could come from multiple fueling locations along the operational path. The methods, systems and computer program products that are described in this disclosure can provide a streamlined model that addresses the convergence of availability and location of supplies, shipment methods (specialized containers), shipment paths (route of distribution) and end point delivery that are also sometimes mobile.

FIG. 9 is a block diagram illustrating a process system 400 that can incorporate the system 100 for two tier distribution optimization that is depicted in FIG. 3. FIG. 9 depicts one embodiment of an exemplary processing system 400 to which the present invention may be applied is shown in accordance with one embodiment. The processing system 400 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102. As illustrated, the system 100 for two tier distribution optimization that can be integrated into the processing system 400 by connection to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 152, 154, and 156 can be the same type of user input device or different types of user input devices. The user input devices 152, 154, and 156 are used to input and output information to and from system 400.

Of course, the processing system 400 may also include other elements (not shown), as readily contemplated by one of skill in the art, as well as omit certain elements. For example, various other input devices and/or output devices can be included in processing system 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 400 are readily contemplated by one of ordinary skill in the art given the teachings of the present invention provided herein.

The present invention may be a system, a method, and/or a computer program product at any possible technical detail level of integration. For example, in some embodiments, a computer program product is provided for system for providing for two tier distribution optimization. The computer program product includes a computer readable storage medium having computer readable program code embodied therewith. The program instructions executable by a processor to cause the processor to receive distribution data for a first item, the distribution data including a starting location and a destination, and the distribution data including a constraint for shipment with a second item. The computer program product can also create, using the processor, a time space network model for tracking the first item relative to the constraint for shipment with the second item. The computer program product can also perform, using the processor, an objective function using the time space network model to optimize the distribution data by minimizing the sum of an objective function including a minimized fulfillment penalty and a minimized unmet order penalty. The computer program product can also execute, using the processor, a delivery plan with the distribution data being optimized.

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

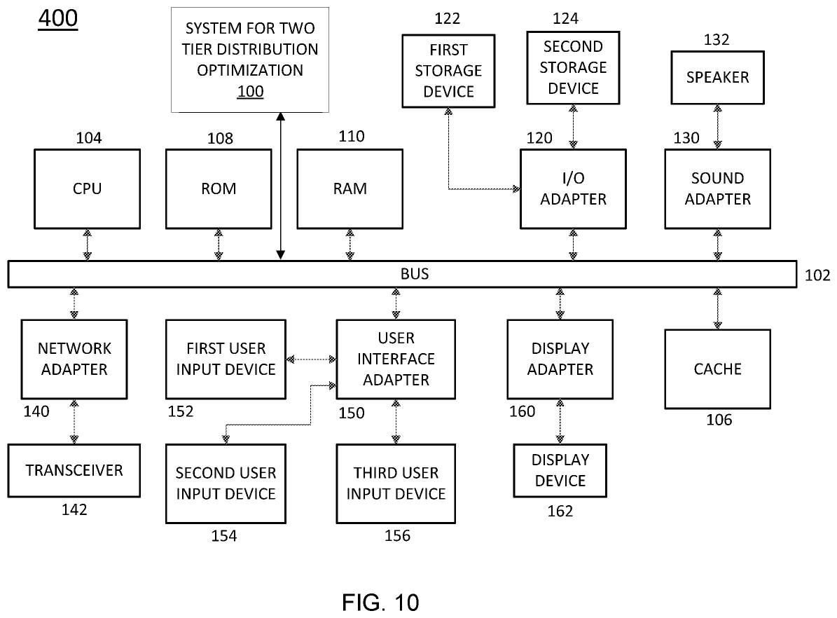

Referring now to FIG. 10, illustrative cloud computing environment 150 is depicted. As shown, cloud computing environment 150 includes one or more cloud computing nodes with which local computing devices used by cloud consumers, such as, for example, personal digital assistant (PDA) or cellular telephone 54A, desktop computer 54B, laptop computer 54C, and/or automobile computer system 54N may communicate. Nodes may communicate with one another. They may be grouped (not shown) physically or virtually, in one or more networks, such as Private, Community, Public, or Hybrid clouds as described hereinabove, or a combination thereof. This allows cloud computing environment 150 to offer infrastructure, platforms and/or software as services for which a cloud consumer does not need to maintain resources on a local computing device. It is understood that the types of computing devices 54A-N shown in FIG. 10 are intended to be illustrative only and that computing nodes and cloud computing environment 150 can communicate with any type of computerized device over any type of network and/or network addressable connection (e.g., using a web browser).

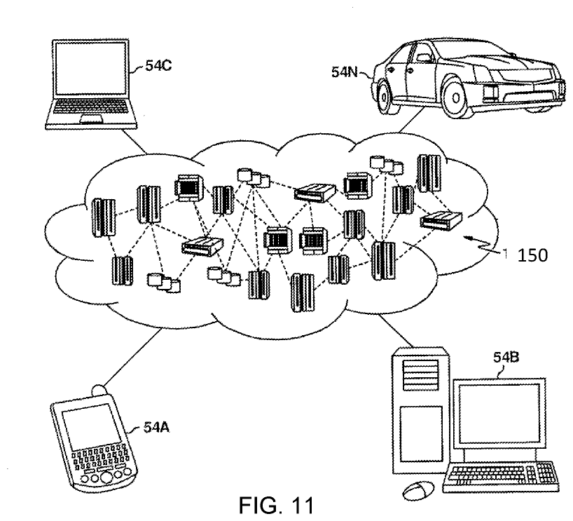

Referring now to FIG. 11, a set of functional abstraction layers provided by cloud computing environment 150 (FIG. 10) is shown. It should be understood in advance that the components, layers, and functions shown in FIG. 11 are intended to be illustrative only and embodiments of the invention are not limited thereto. As depicted, the following layers and corresponding functions are provided:

Hardware and software layer 60 includes hardware and software components. Examples of hardware components include mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and the system 100 that employs visual artefacts including pictograms to search source code.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method for two tier distribution optimization using a time space model which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A computer-implemented method for optimizing distribution of shipping items comprising:
receiving distribution data for a first item, the distribution data including a starting location and a destination, and the distribution data including a constraint for shipment with a second item;
creating a time space network model for tracking the first item relative to the constraint for shipment with the second item;
performing an objective function using the time space network model to optimize the distribution data by minimizing the sum of an objective function including a minimized fulfillment penalty and a minimized unmet order penalty; and
executing a delivery plan with the distribution data being optimized.

2. The computer-implemented method of claim 1, wherein the first item is a vaccine, and the second item is a refrigerated container for shipping the vaccine.

3. The computer-implemented method of claim 1, wherein the time space network includes multiple suppliers and multiple customers, wherein the nodes of the time space network are locations of time and the arcs between the nodes represent movement of the first and second items between the multiple suppliers and multiple customers.

4. The computer-implemented method of claim 3, wherein the arc between nodes includes retaining the second items at a supplier location across multiple nodes of time.

5. The computer-implemented method of claim 1, wherein the first item is a food item, and the second item is a refrigerator container for shipping the food item.

6. The computer implemented method of claim 1, wherein the second item is a shipment method based upon a shipping route.

7. The computer implemented method of claim 1, wherein the first item is a vaccine, and the second item is a refrigerated container for shipping the vaccine, and wherein the creating of the time space network model comprises receiving a master vaccine container plan and a vaccine stock and production plan.

8. The computer implemented method of claim 7, wherein the master vaccine container plan and the vaccine stock and production plan are updated in real time.

9. A system for optimizing distribution of shipping items comprising:
a hardware processor; and
a memory that stores a computer program product, which, when executed by the hardware processor, causes the hardware processor to:
receive distribution data for a first item, the distribution data including a starting location and a destination, and the distribution data including a constraint for shipment with a second item;
create a time space network model for tracking the first item relative to the constraint for shipment with the second item;
perform an objective function using the time space network model to optimize the distribution data by minimizing the sum of an objective function including a minimized fulfillment penalty and a minimized unmet order penalty; and
execute a delivery plan with the distribution data being optimized.

10. The system of claim 9, wherein the first item is a vaccine, and the second item is a refrigerated container for shipping the vaccine.

11. The system of claim 9, wherein the time space network includes multiple suppliers and multiple customers, wherein the nodes of the time space network are locations of time and the arcs between the nodes represent movement of the first and second items between the multiple suppliers and multiple customers.

12. The system of claim 9, wherein the arc between nodes includes retaining the second items at a supplier location across multiple nodes of time.

13. The system of claim 9, wherein the first item is a food item, and the second item is a refrigerator container for shipping the food item.

14. The system of claim 9, wherein the second item is a shipment method based upon a shipping route.

15. A computer program product for optimizing distribution of shipping items comprising a computer readable storage medium having computer readable program code embodied therewith, the program instructions executable by a processor to cause the processor to:
receive, using the processor, distribution data for a first item, the distribution data including a starting location and a destination, and the distribution data including a constraint for shipment with a second item;
create, using the processor, a time space network model for tracking the first item relative to the constraint for shipment with the second item;
perform, using the processor, an objective function using the time space network model to optimize the distribution data by minimizing the sum of an objective function including a minimized fulfillment penalty and a minimized unmet order penalty; and
execute, using the processor, a delivery plan with the distribution data being optimized.

16. The computer program product of claim 15, wherein the first item is a vaccine, and the second item is a refrigerated container for shipping the vaccine.

17. The computer program product of claim 15, wherein the time space network includes multiple suppliers and multiple customers, wherein the nodes of the time space network are locations of time and the arcs between the nodes represent movement of the first and second items between the multiple suppliers and multiple customers.

18. The computer program product of claim 17, wherein the arc between nodes includes retaining the second items at a supplier location across multiple nodes of time.

19. The computer program product of claim 15, wherein the first item is a food item, and the second item is a refrigerator container for shipping the food item.

20. The computer program product of claim 15, wherein the second item is a shipment method based upon a shipping route.
